(19) United States
(12) Patent Application Publication
(10) Pub. No.: US 2019/0060114 A1
(43) Pub. Date: Feb. 28, 2019

(54) Facilitation of Eye Irrigation

(71) Applicant: Ellana Stinson, Quincy, MA (US)

(72) Inventor: Ellana Stinson, Quincy, MA (US)

(21) Appl. No.: 15/687,452

(22) Filed: Aug. 26, 2017

(51) Int. Cl. A61F 9/00; A61M 5/14; A61M 16/06; A61M 39/10

(52) U.S. Cl. CPC A61F 9/0026; A61M 5/14; A61M 16/0666; A61M 39/1011; A61M 2039/1077; A61M 2205/70; A61M 2209/088; A61M 2210/0612; A61M 2210/0618; A61M 2039/1088

(57) ABSTRACT

Eye irrigation can be facilitated via an adapter apparatus to connect an IV tube to a nasal cannula. The adapter apparatus can be constructed to provide a fluid transfer between the IV tube and the nasal cannula. Additionally, a modified nose clip can perform the function of facilitating fluid transfer from an IV bag to the eyes of a patient during an eye irrigation procedure.

### TECHNICAL FIELD

This disclosure relates generally to eye irrigation. More specifically, this disclosure relates to securely connecting an intravenous tube to a nasal cannula.

### BACKGROUND

There are various types of eye irrigation techniques. Irrigation is the application of controlled amounts of water to specific locations at needed intervals. The first line of management for chemical injuries is usually copious irrigation of the eye with an isotonic saline or sterile water. In the cases of chemical burns, one should not try to buffer the solution, but instead dilute it with copious flushing. However, eye irrigation can be a messy process resulting in the dilution fluid in various places other than the eye. Therefore, a means of streamlined and more comfortable eye irrigation is needed to prevent the dilution fluid from wasting or causing pain and discomfort to a patient.

The above-described background relating to eye irrigation is merely intended to provide a contextual overview and is not intended to be exhaustive. Other context regarding the state of the art may become further apparent upon review of the following detailed description.

### SUMMARY

A simplified summary is provided herein to help enable a basic or general understanding of various aspects of exemplary, non-limiting embodiments that follow in the more detailed description and the accompanying drawings. This summary is not intended, however, as an extensive or exhaustive overview. Instead, the purpose of this summary is to present some concepts related to some exemplary non-limiting embodiments in simplified form as a prelude to more detailed descriptions of the various embodiments that follow in the disclosure.

This disclosure comprises a method an apparatus for connecting an intravenous (IV) tube to a nasal cannula.

This disclosure relates to eye irrigation being performed in the field, medical offices, and/or hospitals. The method can allow medical providers to place a patient in a comfortable position for prolonged eye irrigation. In addition, the method and apparatus allows for use of common equipment that most medical facilities keep in stock. Therefore, medical facilities will not need to purchase new equipment bundles or require additional training for use.

Currently, there is no easy way to connect the IV tube to the nasal cannula. Standard IV tubing has an end piece that is designed to screw onto the end of a catheter, via a luer lock, that is placed in the patient's vein. It can be difficult to remove female luer lock piece from the IV tubing in order to securely place the end of the IV tubing into the end of the nasal cannula.

An adapter apparatus can be produced to connect the IV tube and the nasal cannula without mess and hassle. The adapter can have two male ends for insertion into a first female end of the IV tube and a second female end of the nasal cannula. The adapter can facilitate ease of eye irrigation by enabling easy and clean administration of fluid to the eyes using a bag of IV fluid, the IV tubing, and the nasal cannula.

In one embodiment, a first end of the adapter can be cone shaped to facilitate ease of protrusion into the nasal cannula. However, it should be understood that the first end of the adapter can be any shape (e.g., cylindrical, oblong, elliptical, etc.) that can facilitate attachment of the IV tube to the nasal cannula. A second end of the adapter can be cylindrical or cone shaped as well. Either end can also comprise striations for precise fitment into the IV tube and/or the nasal cannula. Alternatively, either end can comprise a cylindrical shaft with helical grooves or ridges called threads around the outside. The aforementioned embodiments can facilitate the adapter to be snuggly connected to a female luer lock piece of the IV tube and/or the nasal cannula.

The method can allow for a user to place the nasal cannula over a patient's nasal bridge and allow water to flow freely from the IV bag through the IV tube, through the adapter, through the nasal cannula, and into the patient's eyes without much effort.

In another embodiment, the disclosure can comprise a modified nasal clamp. This apparatus would provide secure placement of the IV tubing by preventing movement and allowing for a more universal use for all nasal types. For instance, the nasal cannula may not fit snuggly over the nasal bridge of every patient and can require the patient to hold the equipment. Therefore, a properly designed nasal clamp can create an additional level of ease to facilitate eye irrigation.

A tubing similar to a nasal cannula can securely attach to the IV fluid tubing and or an IV bag. The tubing can comprise prongs, similar to those of the nasal cannula, to be positioned on the superior portion of the nasal clamp and be angled towards the medial canthus of each eye. Although this tubing would not require the adapter, a nasal clamp can be adapted to support such a tubing. For instance, the nasal clamp can comprise channels for a tube to be inserted through. The nasal clamp can also comprise a lock for restricting flow of fluid on one side while allowing fluid to flow freely on the other side. For example, if a patient only needs his/her left eye irrigated, then the lock can be utilized on the right side of the nasal clamp to restrict fluid flow from the right tube within the right channel of the nasal clamp, thereby restricting fluid from the right eye of the patient. The lock can be for the right or the left side of the nasal clamp and can be used interchangeably. The opposite end of the tubing (from the nasal clamp) can comprise a female fastening component or the like to facilitate connection with the IV bag.

Described herein are systems, articles of manufacture, and other embodiments or implementations that can facilitate eye irrigation.

According to one embodiment, described herein is an apparatus for facilitating eye irrigation. The apparatus can comprise a first end adapted to connect to a second end of a nasal cannula, wherein the first end comprises a first opening. The apparatus can also comprise a third end adapted to connect to an intravenous fluid tube, wherein the third end comprises a second opening.

According to another embodiment, described herein is a method for facilitating eye irrigation. The method can comprise connecting a first end of an adapter to a second end of a nasal cannula, wherein the connecting the first end of the adapter to the second end of the nasal cannula comprises inserting the first end of the adapter into the second end of the nasal cannula. Furthermore, the method can comprise connecting a third end of the adapter to a fourth end of an intravenous fluid tube, wherein the connecting the third end of the adapter to the fourth end of the intravenous fluid tube comprises screwing a male piece of the intravenous fluid tube into a female piece of the adapter.

According to yet another embodiment, described herein is another apparatus for facilitating eye irrigation. The apparatus can comprise a tube adapted to facilitate fluid to the eye irrigation apparatus, and a nose clamp adapted to attach to a user's nose, wherein the nose clamp comprises a channel for delivering the fluid to an eye of the user.

These and other embodiments or implementations are described in more detail below with reference to the drawings.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1A illustrates an example adapter apparatus for connecting a nasal cannula to an IV fluid tube.

FIG. 1B illustrates an example adapter for connecting a nasal cannula to an IV fluid tube apparatus in use.

FIG. 2A illustrates another example adapter apparatus for connecting a nasal cannula to an IV fluid tube.

FIG. 2B illustrates another example adapter apparatus for connecting a nasal cannula to an IV fluid tube in use.

FIG. 3 illustrates an example modified nasal clamp eye irrigation apparatus in use.

FIG. 4 illustrates is an example system block diagram for a method of using the adapter apparatus for connecting a nasal cannula to an IV fluid tube.

FIG. 5 illustrates is another example system block diagram for another method of utilizing the adapter apparatus for connecting a nasal cannula to an IV fluid tube.

FIG. 6 illustrates yet another example system block diagram for another method of utilizing the adapter apparatus for connecting a nasal cannula to an IV fluid tube.

### DETAILED DESCRIPTION

In the following description, numerous specific details are set forth to provide a thorough understanding of various embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment,” or “an embodiment,” means that a particular feature, structure, or characteristic described in connection with the embodiment is included in at least one embodiment. Thus, the appearances of the phrase “in one embodiment,” “in one aspect,” or “in an embodiment,” in various places throughout this specification are not necessarily all referring to the same embodiment. Furthermore, the particular features, structures, or characteristics may be combined in any suitable manner in one or more embodiments.

The words “exemplary” and/or “demonstrative” are used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

As an overview of the various embodiments presented herein, to correct for the above identified deficiencies and other drawbacks of using carts.

For simplicity of explanation, the methods (or algorithms) are depicted and described as a series of acts. It is to be understood and appreciated that the various embodiments are not limited by the acts illustrated and/or by the order of acts. For example, acts can occur in various orders and/or concurrently, and with other acts not presented or described herein. Furthermore, not all illustrated acts may be required to implement the methods. In addition, the methods could alternatively be represented as a series of interrelated states via a state diagram or events. Additionally, the methods described hereafter are capable of being stored on an article of manufacture (e.g., a computer readable storage medium) to facilitate transporting and transferring such methodologies to computers. The term article of manufacture, as used herein, is intended to encompass a computer program accessible from any computer-readable device, carrier, or media, including a non-transitory computer readable storage medium.

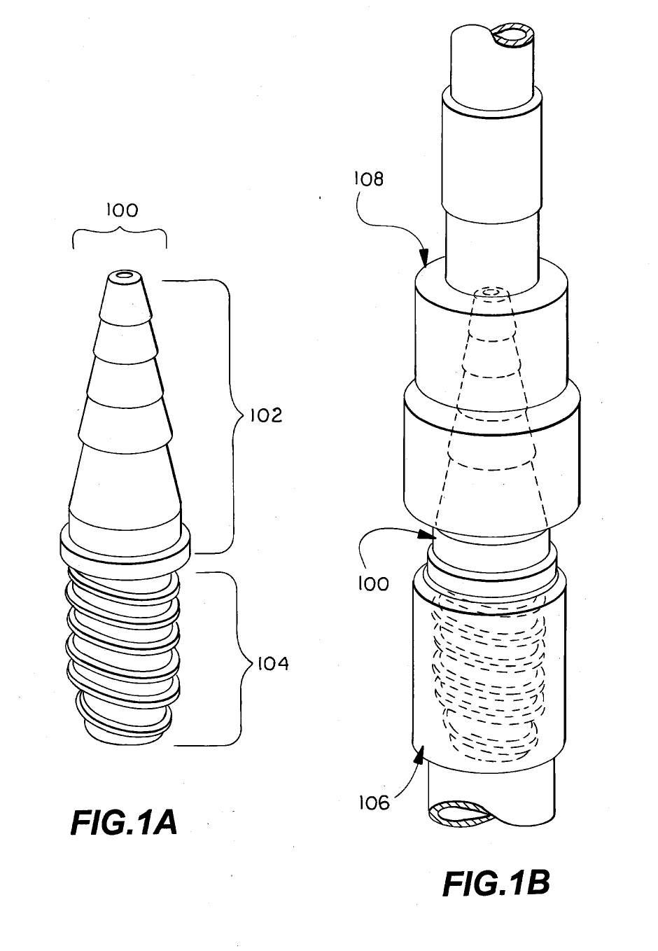

Referring now to FIG. 1A and FIG. 1B, illustrated is an example adapter apparatus for connecting a nasal cannula to an IV fluid tube and the example adapter apparatus in use for connecting a nasal cannula to an IV fluid tube. Adapter 100 can be constructed to facilitate fluid transfer from an IV bag tubing to a nasal cannula. The adapter 100 can comprise two male piece ends of various constructions. For instance, a top male piece end 102 can be constructed in conical shape to facilitate ease of connection with a nasal cannula female piece end 108. The top male piece end 102 can also comprise ridges to prevent the adapter 100 from being easily or accidentally pulled out of nasal cannula tubing. The bottom male piece end 104 can be constructed in a cylindrical shape to facilitate ease of connection with an IV fluid tubing female piece end 106. The bottom male piece end 104 of the adapter 100 can comprise threads to allow the adapter 100 to be screwed into the IV fluid tubing female piece end 106. The top male piece end 102 can then be inserted into the nasal cannula female piece end 108.

It should be noted that these two operations can be performed in either order or simultaneously. It should also be understood that in alternative embodiments, both ends of the adapter 100 can comprise various shapes (e.g., cylindrical, conical, or any shape adapted to facilitate connection of the IV fluid tubing female piece end 106 to the nasal cannula female piece end 108) and/or mechanisms (e.g., the bottom male piece end 104 can be constructed in a snap-and-lock design or a male luer lock design to connect with a female luer lock of the IV fluid tubing female piece end 106) to facilitate connection of the IV tubing to the nasal cannula. Additionally, male pieces of the adapter 100 can be constructed as female pieces to accommodate male pieces of the nasal cannula and/or the IV tube should they be converted from female pieces to male pieces in other embodiments.

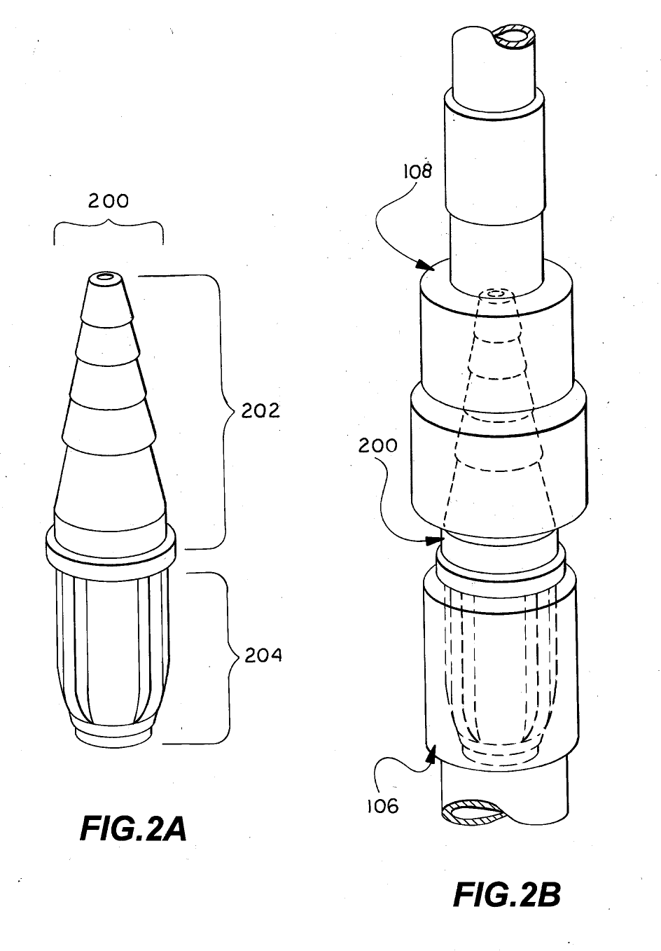

Referring now to FIG. 2A and FIG. 2B, illustrated is an example adapter apparatus for connecting a nasal cannula to an IV fluid tube and an example adapter apparatus in use for connecting a nasal cannula to an IV fluid tube. Adapter 200 can be constructed to facilitate fluid transfer from an IV bag tubing to a nasal cannula. The adapter 200 can comprise two male piece ends of various constructions. For instance, a top male piece end 102 can be constructed in conical shape to facilitate ease of connection with a nasal cannula female piece end 108. The top male piece end 202 can also comprise ridges to prevent the adapter 200 from being easily or accidentally pulled out of nasal cannula tubing. The bottom male piece end 204 can be constructed in a cylindrical shape to facilitate ease of connection with an IV fluid tubing female piece end 106. The bottom male piece end 204 of the adapter 200 can comprise vertical and/or horizontal striations to allow the adapter 200 to be inserted into the IV fluid tubing female piece end 106. The top male piece end 202 can then be inserted into the nasal cannula female piece end 108.

It should be noted that these two operations can be performed in either order or simultaneously. It should also be understood that in alternative embodiments, both ends of the adapter 200 can comprise various shapes (e.g., cylindrical, conical, or any shape adapted to facilitate connection of the IV fluid tubing female piece end 106 to the nasal cannula female piece end 108) and/or mechanisms (e.g., the bottom male piece end 204 can be constructed in a snap-and-lock design or a male luer lock design to connect with a female luer lock of the IV fluid tubing female piece end 106) to facilitate connection of the IV tubing to the nasal cannula.

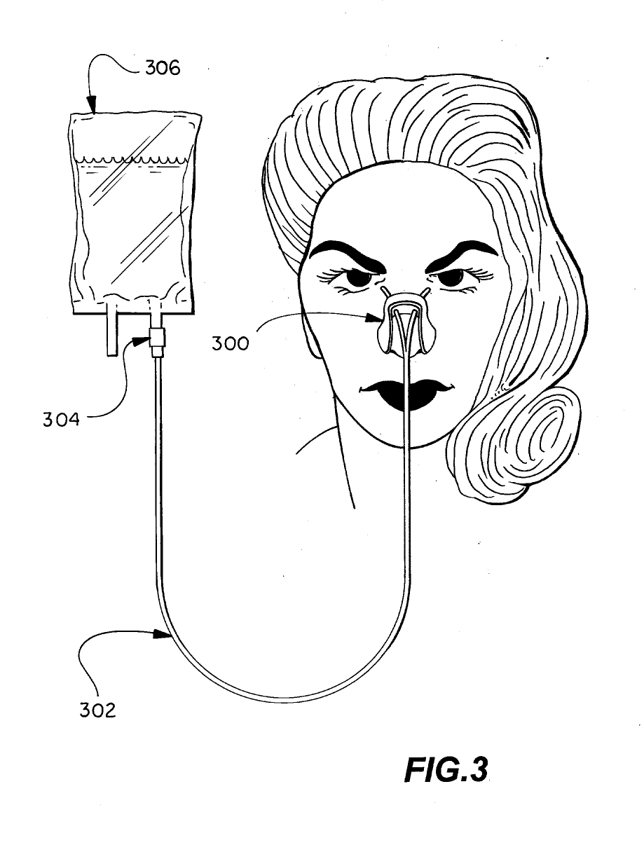

Referring now to FIG. 3, illustrated is an example nasal clamp eye irrigation apparatus in use. A modified nasal clamp 300 can provide secure placement of an IV tube 302 by preventing movement and allowing for a more universal use for all nasal types. The IV tube 302 can be attach directly to an IV bag 306 via an attachment mechanism 304. The attachment mechanism 304 can be any mechanism (e.g., male piece, female piece, lock-and-snap, luer lock, screw threads, etc.) that can be used to attach to an IV bag. The IV tubing 302 can comprise prongs, similar to those of the nasal cannula, to be positioned on the superior portion of the nasal clamp and be angled towards the medial canthus of each eye as depicted in FIG. 3. The prongs of the IV tubing 302 can be created by the IV tubing 302 being split of from one tube into two different tubes, via a juncture at a distal end near the patient. Alternatively, the IV tubing 302 can comprise two separate tubes that are bound together at the attachment mechanism 304, whereby fluid from the IV bag 306 is directly provided to each individual tubing at the attachment mechanism 304.

Although this embodiment utilizing the IV tubing 302 would not require the adapter because it is directly connected to the IV bag 306, a nasal clamp can be adapted to support such a tubing. For instance, the modified nasal clamp 300 can comprise channels for one or more tubes to be inserted through as depicted in FIG. 3. The tubes can be inserted through the front of the modified nasal clamp 300 through the channels, and protrude separately through the modified nasal clamp 300 to form the prongs on the rear end of the modified nasal clamp 300 as depicted in FIG. 3. The nasal clamp can also comprise a lock or blocking mechanism (e.g., valve) for restricting flow of fluid on one side (e.g., prong) of the IV tubing 302 while allowing fluid to flow freely on the other side. For example, if a patient only needs his/her left eye irrigated, then the lock can be utilized on the right side of the modified nasal clamp 300 to restrict or block fluid flow from the right tube of the IV tubing 302 within the right channel of the modified nasal clamp 300, thereby restricting or blocking fluid from the right eye of the patient. The lock can be for the right or the left side of the nasal clamp and they can be used interchangeably, together, or not at all.

The modified nasal clamp 300 can be adapted to adjust to a variety of sizes of patients' noses. For example, a child's nose bridge will generally be smaller than the nose bridge of an adult. Therefore, the modified nasal clamp 300 can be adapted to fit to various users' noses. In an alternate embodiment, the prongs of the modified nasal clamp 300 can be formed of the same material the nose clamp is form of and formed into a ridged structure. In this embodiment, although the tubes can still be inserted through the front of the modified nasal clamp 300 and into the channels of the modified nasal clamp 300, the ridged structure will protrude out of the back end (end closest to the patients eyes) of the modified nasal clamp 300 to form the prongs for fluid dissemination. This structure can comprise an additional channel whereby another type of fluid can be deliver to the patient's eye or eyes during the eye irrigation procedure. For example, a dye can be administered to the patient via the additional channel and the modified nasal clamp 300 can comprise a litmus test (e.g., litmus paper, pH indicator, etc.) to determine the patient's eye condition prior to, during, and after the eye irrigation procedure.

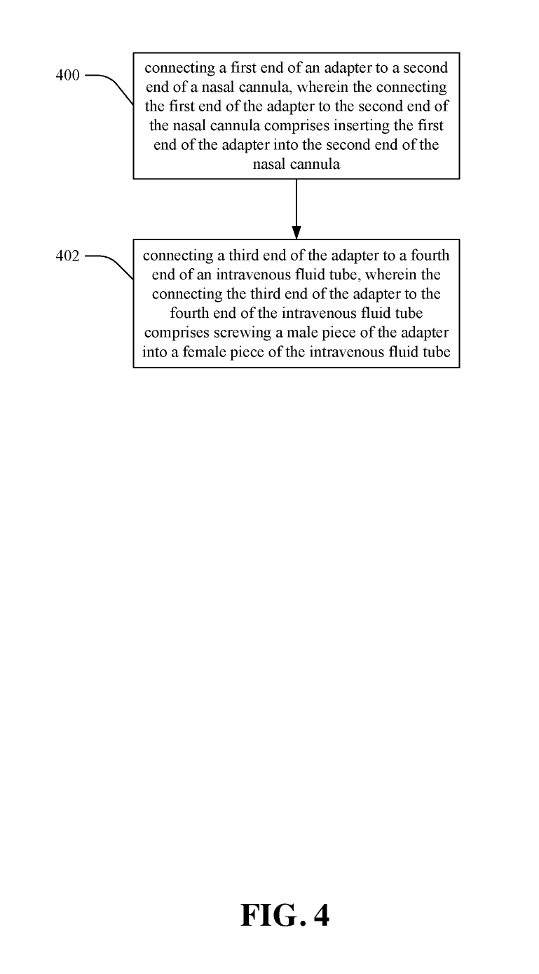

Referring now to FIG. 4, illustrated is an example system block diagram for a method of using the adapter apparatus for connecting a nasal cannula to an IV fluid tube. At element 400, the method comprises connecting a first end of an adapter (e.g. adapter 100) to a second end of a nasal cannula (e.g., nasal cannula female piece end 108), wherein the connecting the first end of the adapter (e.g. adapter 100) to the second end of the nasal cannula (e.g., nasal cannula female piece end 108), wherein the connecting the first end of the adapter (e.g. adapter 100) comprises inserting the first end of the adapter 100 into the second end of the nasal cannula (e.g., nasal cannula female piece end 108).

Additionally, at element 402, the method can comprise connecting a third end of the adapter 100 to a fourth end of an intravenous fluid tube (e.g., IV fluid tubing female piece end 106), wherein the connecting the third end of the adapter 100 to the fourth end of the intravenous fluid tube (e.g., IV fluid tubing female piece end 106) comprises screwing a male piece of the adapter 100 into a female piece of the intravenous fluid tube (e.g., IV fluid tubing female piece end 106).

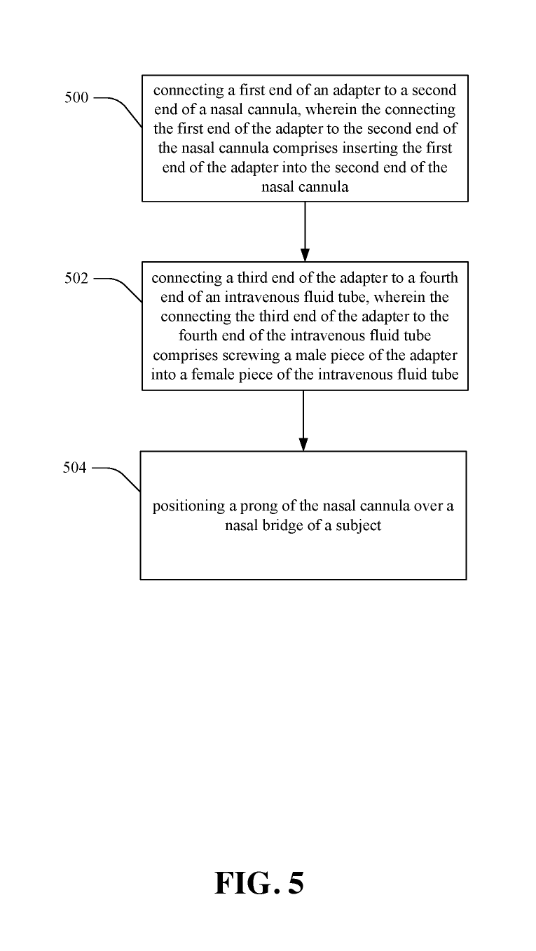

Referring now to FIG. 5, illustrated is another example system block diagram for another method of using the adapter apparatus for connecting a nasal cannula to an IV fluid tube. At element 500, the method comprises connecting a first end of an adapter (e.g. adapter 100) to a second end of a nasal cannula (e.g., nasal cannula female piece end 108), wherein the connecting the first end of the adapter (e.g. adapter 100) to the second end of the nasal cannula (e.g., nasal cannula female piece end 108), wherein the connecting the first end of the adapter (e.g. adapter 100) comprises inserting the first end of the adapter 100 into the second end of the nasal cannula (e.g., nasal cannula female piece end 108).

Additionally, at element 502, the method can comprise connecting a third end of the adapter 100 to a fourth end of an intravenous fluid tube (e.g., IV fluid tubing female piece end 106), wherein the connecting the third end of the adapter 100 to the fourth end of the intravenous fluid tube (e.g., IV fluid tubing female piece end 106) comprises screwing a male piece of the adapter 100 into a female piece of the intravenous fluid tube (e.g., IV fluid tubing female piece end 106). Furthermore, at element 504, the method can comprise positioning a prong of the nasal cannula over a nose bridge of a subject.

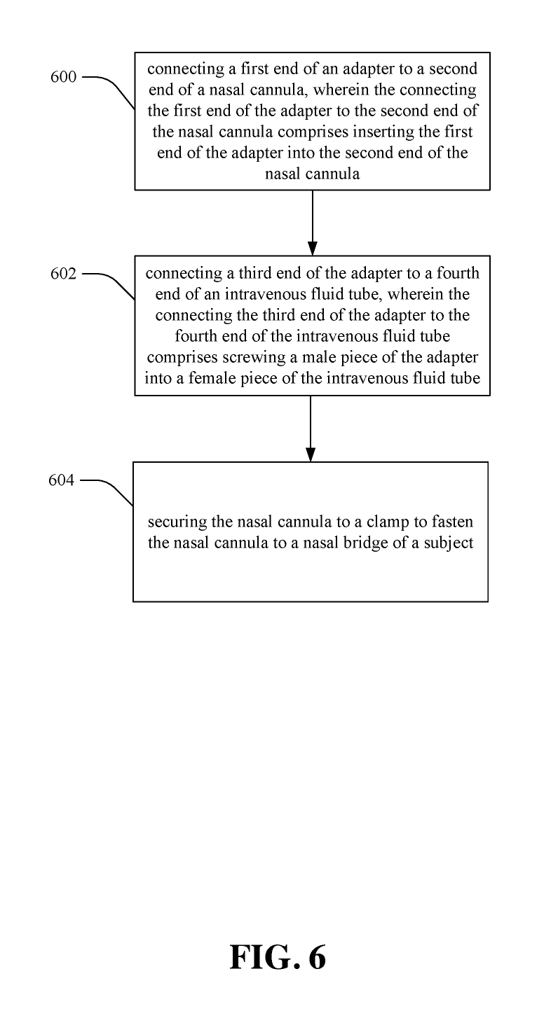

Referring now to FIG. 6, illustrated is yet another example system block diagram for another method of using the adapter apparatus for connecting a nasal cannula to an IV fluid tube. At element 600, the method comprises connecting a first end of an adapter (e.g. adapter 100) to a second end of a nasal cannula (e.g., nasal cannula female piece end 108), wherein the connecting the first end of the adapter (e.g. adapter 100) to the second end of the nasal cannula (e.g., nasal cannula female piece end 108), wherein the connecting the first end of the adapter (e.g. adapter 100) comprises inserting the first end of the adapter 100 into the second end of the nasal cannula (e.g., nasal cannula female piece end 108).

Additionally, at element 602, the method can comprise connecting a third end of the adapter 100 to a fourth end of an intravenous fluid tube (e.g., IV fluid tubing female piece end 106), wherein the connecting the third end of the adapter 100 to the fourth end of the intravenous fluid tube (e.g., IV fluid tubing female piece end 106) comprises screwing a male piece of the adapter 100 into a female piece of the intravenous fluid tube (e.g., IV fluid tubing female piece end 106). Furthermore, at element 604, the method can comprise securing the nasal cannula to a clamp to fasten the nasal cannula to a nasal bridge of a subject..

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding FIGs, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. An adapter apparatus, comprising:
a first end adapted to connect to a second end of a nasal cannula, wherein the first end comprises a first opening; and
a third end adapted to connect to an intravenous fluid tube, wherein the third end comprises a second opening.

2. The adapter apparatus of claim 1, wherein the first end connects to the nasal cannula by fitting within the second end of the nasal cannula.

3. The adapter apparatus of claim 1, wherein the third end further comprises a male luer lock.

4. The adapter apparatus of claim 3, wherein the male luer lock is adapted to connect to the intravenous fluid tube via a female luer lock of the intravenous fluid tube.

5. The adapter apparatus of claim 1, wherein the first end is cone shaped.

6. The adapter apparatus of claim 1, wherein the first end is adapted to connect to the second end of the nasal cannula via a snap and lock system.

7. The adapter apparatus of claim 1, wherein the third end is adapted to connect to the intravenous fluid tube via a snap and lock system.

8. The adapter apparatus of claim 1, wherein the first end is cylinder shaped.

9. A method, comprising:
connecting a first end of an adapter to a second end of a nasal cannula, wherein the connecting the first end of the adapter to the second end of the nasal cannula comprises inserting the first end of the adapter into the second end of the nasal cannula; and
connecting a third end of the adapter to a fourth end of an intravenous fluid tube, wherein the connecting the third end of the adapter to the fourth end of the intravenous fluid tube comprises screwing a male piece of the adapter into a female piece of the intravenous fluid tube.

10. The method of claim 9, wherein the male piece of the adapter comprises a male luer lock.

11. The method of claim 9, further comprising:
positioning a prong of the nasal cannula over a nasal bridge of a subject.

12. The method of claim 9, further comprising:
securing the nasal cannula to a clamp to fasten the nasal cannula to a nasal bridge of a subject.

13. An eye irrigation apparatus, comprising:
a tube adapted to facilitate a fluid to the eye irrigation apparatus; and
a nose clamp adapted to attach to a user's nose, wherein the nose clamp comprises a channel for delivering the fluid to an eye of a user.

14. The apparatus of claim 13, wherein the channel comprises a rigid structure.

15. The apparatus of claim 13, wherein the channel is a first channel, wherein the eye is a first eye, and wherein the nose clamp comprises a second channel to deliver the fluid to a second eye of the user.

16. The apparatus of claim 15, wherein the nose clamp comprises a valve to restrict the fluid from being delivered to the second eye of the user as the fluid is being delivered to the first eye to the user.

17. The apparatus of claim 13, wherein the nose clamp is adapted to adjust to a size of the user's nose.

18. The apparatus of claim 13, wherein the tube comprises a male luer lock adapted to connect to a female luer lock of an intravenous fluid tube.

19. The apparatus of claim 14, wherein the rigid structure comprises litmus test.

20. The apparatus of claim 16, wherein the fluid is a first fluid and wherein the nose clamp comprises a third channel to deliver a second fluid to the first channel.
